(19) United States
(12) Patent Application Publication
(10) Pub. No.: US 2006/0015124 A1
(43) Pub. Date: Jan. 19, 2006

(54) Device for removal treatment of hard skin

(76) Inventor: Armin Floerke, Obertshausen (DE)

Correspondence Address: THE WEBB LAW FIRM, P.C., 700 KOPPERS BUILDING, 436 SEVENTH AVENUE, PITTSBURGH, PA, 15219, US

(21) Appl. No.: 11/176,949

(22) Filed: Jul. 7, 2005

(30) Foreign Application Priority Data
Jul. 7, 2004 (DE) 10 2004 032 956.7

(51) Int. Cl. A61B 17/50

(52) U.S. Cl. 606/131

(57) ABSTRACT

The invention relates to a device for removal treatment of hard skin, comprising a handle region and a first and a second tool region. The invention provides that the first tool region on the one hand and the second tool region on the other hand are arranged at opposing ends of a connecting element, wherein the handle region—enclosing the connecting element in an encasing fashion—is constructed so that it at least partly overlaps the first or the second tool region as desired and is displaceably but fixedly mounted on the connecting element.

The invention relates to a device for the removal treatment of hard skin according to the preamble of claim 1.

A device of the type described initially is known from DE 33 20 594 C2. This comprises a handle region, a first tool region embodied as a hard skin shaver and a second tool region embodied as a hard skin scraper. As required, either the hard skin shaver as such is used (in this case the scraper region embodied as a separate component can be placed aside) or the scraper region is affixed at the blade area of the hard skin shaver using a snap closure and specifically in a manner such that only the scraper region can then be used since the blade of the hard skin shaver is covered thereby.

Whereas hard skin is specifically shaved off in layers using a hard skin shaver—in practice as with a razor, a scraper is used to remove the hard skin bluntly as with a file. The shaver produces flaps of hard skin whereas the scraper produces non-uniform fragments of hard skin.

An advantage of the hard skin shaver according to DE 33 20 594 C2 is that both the shaver and the scraper functions are available as required. A disadvantage however is that in order to change between the functions, a certain “conversion requirement” exists, that is to snap the scraper region on to the shaver region.

It is the object of the invention to improve a device of the type specified initially in that the effort for changing from one tool to the other is still lower.

This object is solved with a device of the type specified initially by the features specified in the characterising part of claim 1.

According to the invention it is thus provided that the first tool region on the one hand and the second tool region on the other hand are arranged at opposing ends of a connecting element, wherein the handle region—enclosing the connecting element in an encasing fashion—is constructed so that it at least partly overlaps the first or the second tool region as desired and is displaceably but fixedly mounted on the connecting element.

In other words, it is provided according to the invention to arrange both tool regions at opposing ends of a connecting element, these being enclosed by a casing section displaceable thereon which at the same time serves as a handle region. As required, the casing-like handle region covers the first or the second tool region as desired whilst conversely the second or the first tool region can be used for the removal treatment of hard skin.

Other advantageous further developments are obtained from the dependent claims.

The device according to the invention including its advantageous further developments according to the dependent claims will be explained in detail hereinafter with reference to the drawings showing two preferred exemplary embodiments.

In the figures

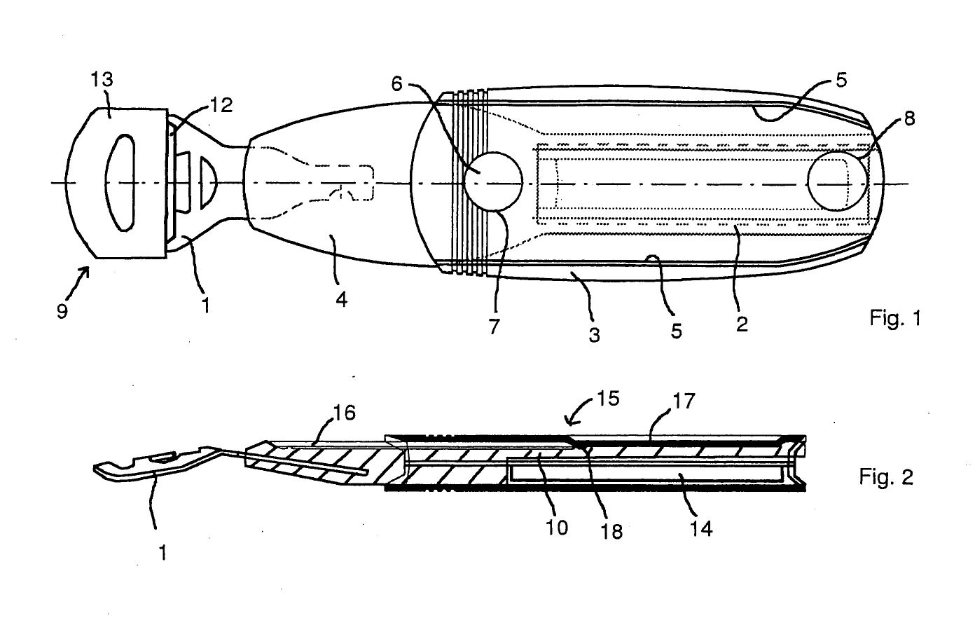

FIG. 1 is a plan and partly cutaway view of the device according to the invention in hard skin shaver mode,

FIG. 2 is a side view and mostly sectional view of the device according to FIG. 1 without the blade and cover plate on the hard skin shaver;

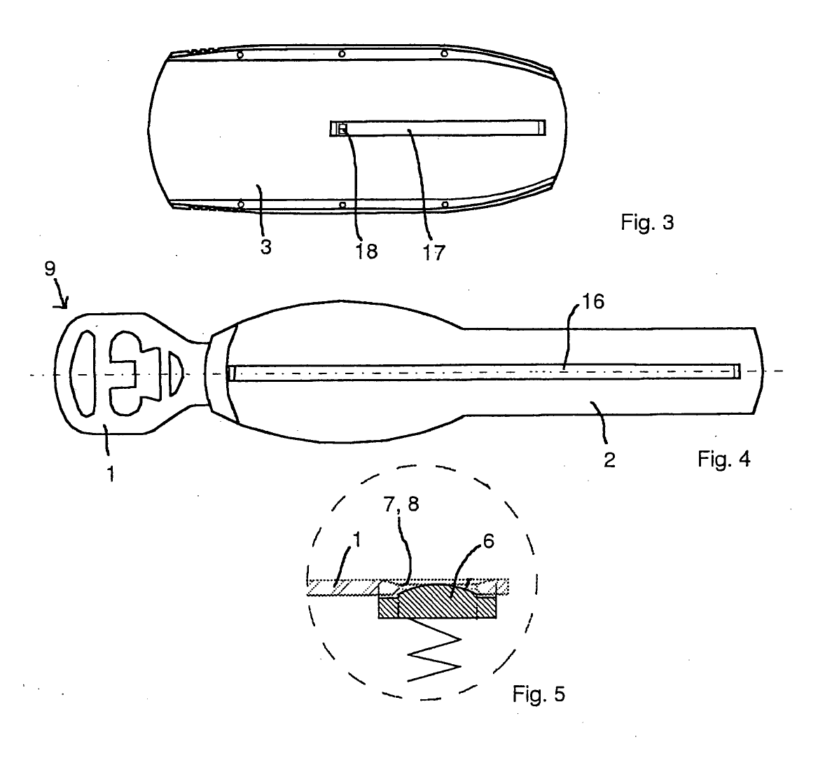

FIG. 3 shows half of the handle region with a guide element embodied as a bead;

FIG. 4 is a view from below of the device according to FIG. 1 with the handle region dismounted;

FIG. 5 is a sectional view of the locating element embodied as a spring-loaded push-button together with the counter-locating element embodied as a recess;

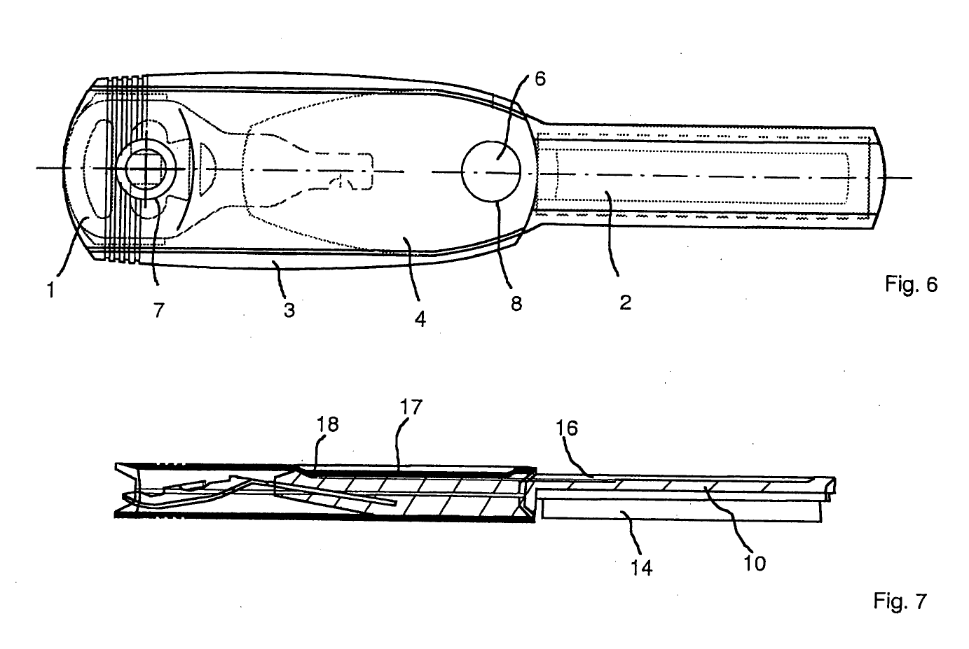

FIG. 6 shows a plan and partly cutaway view of the device according to FIG. 1 in hard skin scraper mode;

FIG. 7 is a side view and mostly sectional view of the device according to FIG. 6;

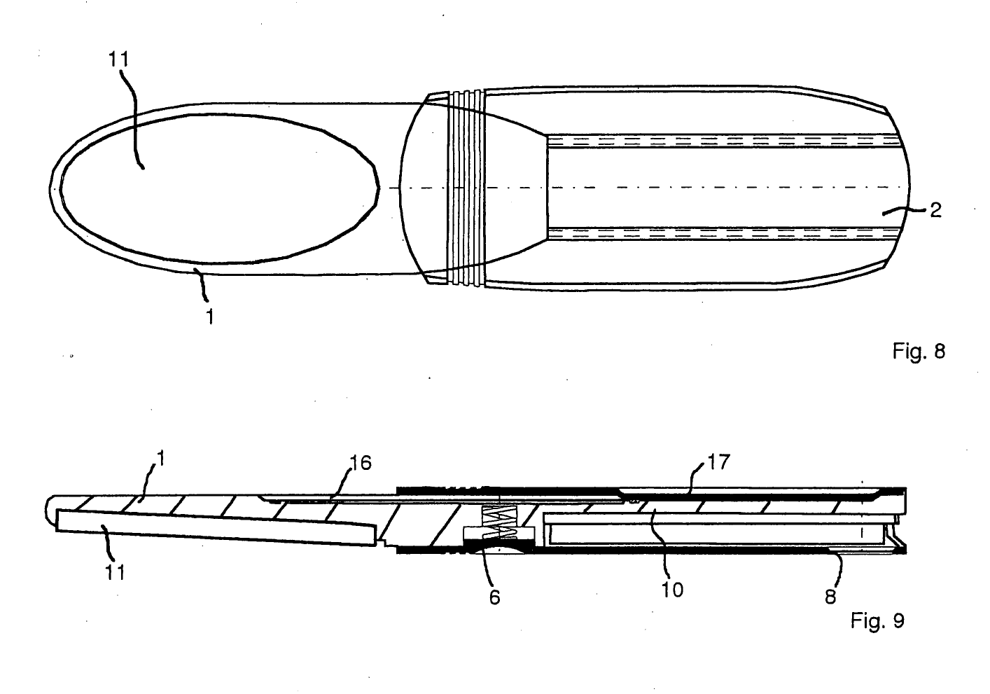

FIG. 8 shows a plan and partly cutaway view of the device according to the invention with mineral filing stone and hard skin scraper; and

FIG. 9 is a side cutaway view of the device according to FIG. 8.

FIGS. 1 to 7 show a first preferred embodiment of the device according to the invention for removal treatment of hard skin. Said device comprises a handle region 3, a first tool region 1 embodied as a hard skin shaver and a second tool region 2 embodied as a hard skin scraper. In this respect, said device does not differ from the device according to the aforesaid DE 33 20 594 C2, where, as will be explained in detail further below with reference to FIGS. 8 and 9, mineral filing stones or the like can also be considered as tools in principle.

For the device according to the invention it is now important that the first tool region 1 (in this case a hard skin shaver) on the one hand and the second tool region 2 (in this case a hard skin scraper) on the other hand are arranged at opposing ends of a connecting element 4, wherein the handle region 3—enclosing the connecting element 4 in a encasing fashion—is constructed so that it at least partly overlaps the first 1 or the second 2 tool region as desired and is displaceably but fixedly mounted on the connecting element 4.

As can easily be seen from FIGS. 1 to 7, the function of the device can thus be adjusted by displacing the handle region 3. In FIG. 1 the tool region 1 serves as a hard skin shaver whereas the tool region 2 embodied as a hard skin scraper is covered by the handle region 3.

As in DE 33 20 594 C2, it is preferably provided according to FIG. 1 that a blade region 9 forming the hard skin shaver is constructed of metal and has a blade 12 and a cover plate 13 for fixing the preferably arched blade 12.

In FIG. 6 on the other hand, the hard skin shaver is covered by the handle region 3 and the device serves as a hard skin scraper with the second tool region 2. In this case, it is preferably provided that a scraper region 10 forming the second tool region 2 (see FIG. 7), provided with a preferably metal and arched scraper plate 14, is constructed as an extended oblong, that is as in DE 33 20 594 C2.

With regard to the materials used, it is advantageously provided that the handle region 3, the connecting element 4 and the scraper region 10 are formed of plastic. In this way, the device can easily be manufactured by injection moulding for example. The blade or scraper plate usually (but not necessarily) consists of metal.

In order that the handle region 3 can easily be displaced on the connecting element 4, according to the embodiment shown it is advantageously provided that the casing-like handle region 3 has an inner contour matched to the outer contour of the connecting element 4. Furthermore, in order to form a stop limitation, in any case in one direction of displacement, it is additionally advantageously provided that both the outer contour of the connecting element 4 and also at least the inner contour of the handle region 3 are constructed as tapering as desired to the first 1 or second 2 tool region. FIGS. 1 to 7 show the second of two said variants; however, the first can easily be imagined wherein it should merely be noted that in this case, the first tool region 1 must be narrower than the tapered opening on the handle region 3.

To guide the connecting element 4 inside the handle region 3, it is advantageously provided that its inner contour has guides 5 running at least partly parallel. The definition “at least partly” expresses the fact that the parallel guidance extends in any case up to the point where the afore-mentioned tapering of the handle region 3 is possibly arranged.

In order to achieve the afore-mentioned fixability of the handle region 3 in the two end positions, it is advantageously provided that a locating element 6 is arranged on the connecting element 4 which can alternately engage in at least two counter-locating elements 7, 8 arranged on the handle region 3.

According to FIG. 5, for this purpose the locating element 6 is preferably embodied as a spring-loaded push-button which fits to the counter-locating elements 7, 8 in positive contact. The counter-locating elements 7, 8 are then, as shown, preferably embodied as round recesses and arranged at both ends of the handle region 3.

In order to further improve the guidance of the connecting element 4 in the handle region 3, it is advantageously provided that that a guide element running along the device is provided, said element being matched to the inner contour of the handle region 3 and also matched to the outer contours of the extended-oblong second tool region 2 and the connecting element 4. More precisely, such a guide element 15 is provided which is embodied as a groove 16 arranged on the second tool region 2 and the connecting element 4 with a corresponding bead 17 which slides along correspondingly therein and is arranged on the inside of the handle region 3. In this situation this guide element 15 can ultimately advantageously be used for delimiting the displacement of the connecting element 4 inside the handle region 3 and thus as a stop or locating element 18 and specifically by suitably dimensioning the lengths of groove 16 and bead 17.

Finally, FIGS. 8 and 9 show a second preferred embodiment where the first tool region 1 is embodied as a mineral filing stone 11 known per se. Such a file is also used to remove hard skin but is usually less abrasive than the hard skin scraper because of the relatively fine grain sizes. In this embodiment the function as scraper or here as file is also defined by displacing the handle region 3.

Reference List

1 Tool region
2 Tool region
3 Handle region
4 Connecting element
5 Guide
6 Locating element
7 Counter-locating element
8 Counter-locating element
9 Blade region
10 Scraper region
11 Mineral filing stone
12 Blade
13 Cover plate
14 Scraper plate
15 Guide element
16 Groove
17 Bead
18 Locating element

1. A device for removal treatment of hard skin, comprising a handle region and a first and a second tool region, wherein the first tool region on the one hand and the second tool region on the other hand are arranged at opposing ends of a connecting element, wherein the handle region—enclosing the connecting element in an encasing fashion—is constructed so that it at least partly overlaps the first or the second tool region as desired and is displaceably but fixedly mounted on the connecting element.

2. The device according to claim 1, wherein the handle region, constructed in an encasing fashion, has an inner contour matched to the outer contour of the connecting element.

3. The device according to claim 2, wherein both the outer contour of the connecting element and also at least the inner contour of the handle region are constructed as tapering towards the first or the second tool region as desired.

4. The device according to claim 2, wherein the inner contour of the handle region has at least partly parallel guides for displaceable mounting of on the connecting element.

5. The device according to claim 1 wherein a locating element is arranged on the connecting element, said locating element, which can alternately engage in at least two counter-locating elements arranged on the handle region.

6. The device according to claim 5, wherein the locating element is constructed as a spring-loaded push-button which fits to the counter-locating elements in positive contact.

7. The device according to claim 5, wherein the counter-locating elements are embodied as round recesses and are arranged at both ends of the handle region.

8. The device according to claim 2, wherein a guide element running along the device is provided, said element being matched to the inner contour of the handle region and also matched to the outer contours of the first or second tool regions and the connecting element.

9. The device according to claim 8, wherein the guide element is embodied as a groove with a corresponding bead which slides along therein.

10. The device according to claim 8, wherein the guide element comprises a locating element for delimiting the displacement of the connecting element inside the handle region.

11. The device according to claim 1, wherein the handle region, the connecting element and at least one of the first and second tool regions are made of plastic.

12. The device according claim 1, wherein the tool regions are embodied as hard skin shavers with a blade region, hard skin scrapers with a scraper region or mineral files with mineral filing stone.

13. The device according to claim 12, wherein the blade region is constructed of one of metal and plastic and has a metal blade and a cover plate for fixing the blade.

14. The device according to claim 12, wherein the scraper region, provided with a scraper plate, is constructed as an extended oblong.
